(19) United States
(12) Patent Application Publication
(10) Pub. No.: US 2010/0331679 A1
(43) Pub. Date: Dec. 30, 2010

(54) PULSED ECHO SENSING DEVICE AND METHOD FOR AN ORTHOPEDIC JOINT

(75) Inventor: Marc T. Stein, Chandler, AZ (US)

Correspondence Address: Orthosensor, Inc., 1560 Sawgrass Corporate Pkwy, 4th Floor, Sunrise, FL, 33323, US

(73) Assignee: OrthoSensor, Sunrise, FL (US)

(21) Appl. No.: 12/748,064

(22) Filed: Mar. 26, 2010

Related U.S. Application Data
(60) Provisional application No. 61/221,901, filed on Jun. 30, 2009.
(60) Provisional application No. 61/221,761, filed on Jun. 30, 2009.
(60) Provisional application No. 61/221,767, filed on Jun. 30, 2009.
(60) Provisional application No. 61/221,799, filed on Jun. 30, 2009.
(60) Provisional application No. 61/221,788, filed on Jun. 30, 2009.
(60) Provisional application No. 61/221,793, filed on Jun. 30, 2009.
(60) Provisional application No. 61/221,801, filed on Jun. 30, 2009.
(60) Provisional application No. 61/221,808, filed on Jun. 30, 2009.
(60) Provisional application No. 61/221,817, filed on Jun. 30, 2009.
(60) Provisional application No. 61/221,867, filed on Jun. 30, 2009.
(60) Provisional application No. 61/221,874, filed on Jun. 30, 2009.
(60) Provisional application No. 61/221,879, filed on Jun. 30, 2009.
(60) Provisional application No. 61/221,881, filed on Jun. 30, 2009.
(60) Provisional application No. 61/221,886, filed on Jun. 30, 2009.
(60) Provisional application No. 61/221,889, filed on Jun. 30, 2009.
(60) Provisional application No. 61/221,894, filed on Jun. 30, 2009.
(60) Provisional application No. 61/221,901, filed on Jun. 30, 2009.
(60) Provisional application No. 61/221,909, filed on Jun. 30, 2009.
(60) Provisional application No. 61/221,916, filed on Jun. 30, 2009.
(60) Provisional application No. 61/221,923, filed on Jun. 30, 2009.
(60) Provisional application No. 61/221,929, filed on Jun. 30, 2009.

(51) Int. Cl. A61B 8/00

(52) U.S. Cl. 600/437

(57) ABSTRACT

At least one embodiment is directed to a sensor for measuring a skeletal system. A signal path of the system comprises an amplifier (612), a sensor element, and an amplifier (620). The sensor element comprises a transducer (4), a waveguide (5), and a reflecting surface (30). An external condition is applied to the sensor element. For example, the sensor element is placed in an artificial orthopedic joint to measure loading of the joint. Pulsed energy waves are emitted by the transducer (4) into the waveguide (5) and the reflected back to be received by the transducer (4). The transit time of each pulsed energy wave corresponds to the external condition applied to the sensor. The transducer (4) outputs a signal corresponding to each pulsed energy wave. A detection circuit edge detects the signal and outputs a pulse to the transducer (4) to generate a new pulse energy wave.

### CROSS-REFERENCE TO RELATED APPLICATIONS

This application claims the priority benefit of U.S. provisional patent applications No. 61/221,761, 61/221,767, 61/221,779, 61/221,788, 61/221,793, 61/221,801, 61/221,808, 61/221,817, 61/221,867, 61/221,874, 61/221,879, 61/221,881, 61/221,886, 61/221,889, 61/221,894, 61/221,901, 61/221,909, 61/221,916, 61/221,923, and 61/221,929 all filed 30 Jun. 2009. The disclosures of which are incorporated herein by reference in its entirety.

### FIELD

The invention relates in general to orthopedics, and particularly though not exclusively, is related to measuring a parameter of a mammalian joint.

### BACKGROUND

The skeletal system of a mammal is subject to variations among species. Further changes can occur due to environmental factors, degradation through use, and aging. An orthopedic joint of the skeletal system typically comprises two or more bones that move in relation to one another. Movement is enabled by muscle tissue and tendons attached to the skeletal system of the joint. Ligaments hold and stabilize the one or more joint bones positionally. Cartilage is a wear surface that prevents bone-to-bone contact, distributes load, and lowers friction.

There has been substantial growth in the repair of the human skeletal system. In general, orthopedic joints have evolved as information from simulations, mechanical prototypes, and long-term patient joint replacement data is collected and used to initiate improved designs. Similarly, the tools being used for orthopedic surgery have been refined over the years but have not changed substantially. Thus, the basic procedure for replacement of an orthopedic joint has been standardized to meet the general needs of a wide distribution of the population. Although the tools, procedure, and artificial joint meet a general need, each replacement procedure is subject to significant variation from patient to patient. The correction of these individual variations relies on the skill of the surgeon to adapt and fit the replacement joint using the available tools to the specific circumstance.

### BRIEF DESCRIPTION OF THE DRAWINGS

Exemplary embodiments will become more fully understood from the detailed description and the accompanying drawings, wherein:

FIG. 1 is an illustration of a sensor placed in contact between a femur and a tibia for measuring a parameter in accordance with an exemplary embodiment;

FIG. 2 is a simplified cross-sectional view of a sensing module (or assemblage) in accordance with an exemplary embodiment;

FIG. 3 is an exemplary assemblage for illustrating reflectance and unidirectional modes of operation;

FIG. 4 is an exemplary assemblage that illustrates propagation of ultrasound waves within a waveguide in the bi-directional mode of operation of this assemblage;

FIG. 5 is an exemplary cross-sectional view of a sensor element to illustrate changes in the propagation of ultrasound waves with changes in the length of a waveguide;

FIG. 6 is an exemplary block diagram of a measurement system in accordance with an exemplary embodiment; and

FIG. 7 is measurement system operating in a pulsed echo mode with digital output according to one embodiment.

### DETAILED DESCRIPTION OF EXEMPLARY EMBODIMENTS

The following description of exemplary embodiment(s) is merely illustrative in nature and is in no way intended to limit the invention, its application, or uses.

Processes, techniques, apparatus, and materials as known by one of ordinary skill in the art may not be discussed in detail but are intended to be part of the enabling description where appropriate. For example specific computer code may not be listed for achieving each of the steps discussed, however one of ordinary skill would be able, without undo experimentation, to write such code given the enabling disclosure herein. Such code is intended to fall within the scope of at least one exemplary embodiment.

Additionally, the sizes of structures used in exemplary embodiments are not limited by any discussion herein (e.g., the sizes of structures can be macro (centimeter, meter, and larger sizes), micro (micrometer), nanometer size and smaller).

Notice that similar reference numerals and letters refer to similar items in the following figures, and thus once an item is defined in one figure, it may not be discussed or further defined in the following figures.

In all of the examples illustrated and discussed herein, any specific values, should be interpreted to be illustrative only and non-limiting. Thus, other examples of the exemplary embodiments could have different values.

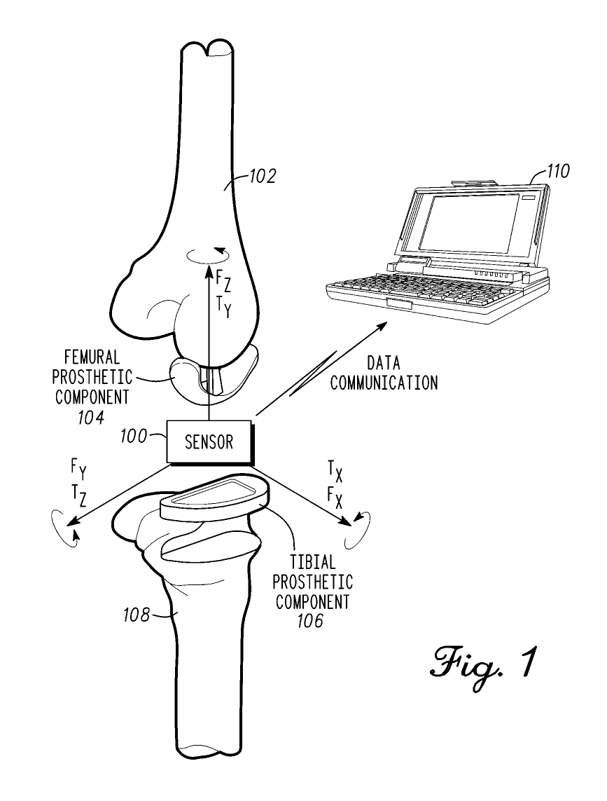

FIG. 1 is an illustration of a sensor 100 placed in contact between a femur 102 and a tibia 108 for measuring a parameter in accordance with an exemplary embodiment. In general, sensor 100 is placed in or in proximity to a feature of the skeletal system. In non-limiting example, sensor 100 is placed within an artificial joint coupled to two or more bones of a skeletal system that move in relation to one another. Embodiments of sensor 100 are broadly directed to measurement of physical parameters, and more particularly, to evaluating changes in the transit time of a pulsed energy wave propagating through a medium. In-situ measurements during orthopedic joint implant surgery would be of substantial benefit to verify an implant is in balance and under appropriate loading. In one embodiment, the instrument is similar to and operates familiarly with other instruments currently used by surgeons. This will increase acceptance and reduce the adoption cycle for a new technology. The measurements will allow the surgeon to ensure that the replacement implant is within predetermined ranges that maximize working life of the joint and minimize rework. Joint implants will become more consistent from surgeon to surgeon. A further issue is that there is little or no implant data generated from the implant surgery, post-operatively, and long term. Sensor 100 can provide implant status data to the orthopedic manufacturers and surgeons. Moreover, data generated by direct measurement of the implanted joint itself would greatly improve the knowledge of implanted joint operation and joint wear thereby leading to improved design and materials.

In at least one exemplary embodiment, an energy pulse is directed within one or more waveguides in sensor 100 by way of pulse mode operations and pulse shaping. The waveguide is a conduit that directs the energy pulse in a predetermined direction. The energy pulse is typically confined within the waveguide. In one embodiment, the waveguide comprises a polymer material. For example, urethane or polyethylene are polymers suitable for forming a waveguide. In one embodiment, the polymer waveguide can be compressed and has little or no hysteresis in the system. A transit time of an energy pulse through a medium is related to the material properties of the medium. This relationship is used to generate accurate measurements of parameters such as distance, weight, strain, pressure, wear, vibration, and density to name but a few.

Sensor 100 can be size constrained by form factor requirements of fitting in a region of a joint of the skeletal system. In one embodiment, sensor 100 can be fitted in a tool having a surface exposed or coupled for measuring a parameter of the muscular-skeletal system. The mechanical portion of sensor 100 comprises a stack of a first transducer, a medium, and an acoustically reflective surface. In a non-limiting example, sensor 100 is used to aid to adjust and balance a replacement knee joint. A knee prosthesis comprises a femoral prosthetic component 104, an insert, and a tibial prosthetic component 106. A distal end of femur 102 is prepared and receives femoral prosthetic component 104. Femoral prosthetic component 104 typically has two condyle surfaces that mimic a natural femur. As shown, femoral prosthetic component 104 has single condyle surface being coupled to femur 100. Femoral prosthetic component 104 is typically made of a metal or metal alloy.

A proximal end of tibia 108 is prepared to receive tibial prosthetic component 106. Tibial prosthetic component 106 is a support structure that is fastened to the proximal end of the tibia and is usually made of a metal or metal alloy. The tibial prosthetic component 106 also retains the insert in place fixed in position to tibia 108. The insert is fitted between femoral prosthetic component 104 and tibial prosthetic component 106. The insert has at least one bearing surface that is in contact with at least condyle surface of femoral prosthetic component 104. The condyle surface can move in relation to the bearing surface of the insert such that the lower leg can rotate under load. The insert is typically made of a high wear plastic material that minimizes friction.

In a knee joint replacement process, the surgeon affixes femoral prosthetic component 104 to the femur 102 and tibial prosthetic component 106 to tibia 108. The tibial prosthetic component 106 can include a tray or plate affixed to the planarized proximal end of the tibia 108. Sensor 100 is placed between a condyle surface of femoral prosthetic component 104 and a major surface of tibial prosthetic component 106. Sensor 100 can be a trial insert that is subsequently removed after measurements are taken in one or more leg positions. Alternatively, sensor 100 can be integrated into an insert for taking measurements. The condyle surface contacts a major surface of sensor 100. The major surface of sensor 100 approximates a surface of the insert. Tibial prosthetic component 106 can include a cavity on the major surface that receives and retains sensor 100 during a measurement process. Tibial prosthetic component 106 and sensor 100 has a combined thickness that represents a combined thickness of tibial prosthetic component 106 and a final insert of the knee joint.

In one embodiment, two sensors 100 are fitted into two separate cavities of tibial prosthetic component 106. Each sensor is independent and each measures a respective condyle of femur 102. Separate sensors also accommodate a situation where a single condyle is repaired and only a single sensor is used. Alternatively, the electronics can be shared between two sensors to lower cost and complexity the circuitry of which will be disclosed in more detail hereinbelow. The shared electronics can multiplex between each sensor module to take measurements when appropriate. Measurements taken by sensor 100 aid the surgeon in modifying the absolute loading on each condyle and the balance between condyles. Although shown for a knee implant, sensor 100 can be used to measure other orthopedic joints such as the spine, hip, shoulder, elbow, ankle, wrist, interphalangeal joint, metatarsophalangeal joint, metacarpophalangeal joints, and others. Alternatively, sensor 100 can be adapted to orthopedic tools to provide measurements.

The prosthesis incorporating sensor 100 emulates the function of a natural knee joint. Sensor 100 can measure loads or other parameters at various points throughout the range of motion. Data from sensor 100 is transmitted to a receiving station 110 via wired or wireless communications. In a first embodiment, sensor 100 is a disposable system. After using sensor 100 to optimally fit the joint implant, it can be disposed of after the operation is completed. Sensor 100 is a low cost disposable system that reduces capital expenditures, maintenance, and accounting when compared to other measurement systems. In a second embodiment, a methodology can be put in place to clean and reuse sensor 100. In a third embodiment, sensor 100 can be incorporated in a tool instead of being a component of the replacement joint. The tool can be disposable or be cleaned for reuse. In a fourth embodiment, sensor 100 can be a permanent component of the replacement joint. Sensor 100 can be used to provide both short term and long term post-operative data on the implanted joint. The receiving station 110 can include data processing, storage, or display, or combination thereof and provide real time graphical representation of the level and distribution of the load. Receiving station 110 can record and provide accounting information of sensor 100 to an appropriate authority.

In an intra-operative example, sensor 100 can measure forces (Fx, Fy, Fz) with corresponding locations and torques (e.g. Tx, Ty, and Tz) on the femoral prosthetic component 104 and the tibial prosthetic component 106. The measured force and torque data is transmitted to receiving station 110 to provide real-time visualization for assisting the surgeon in identifying any adjustments needed to achieve optimal joint pressure and balancing. The data has substantial value in determining ranges of load and alignment tolerances required to minimize rework and maximize longevity of the joint.

As mentioned previous sensor 100 can be used for other joint surgeries; it is not limited to knee replacement implant or implants. Moreover, sensor 100 is not limited to trial measurements. Sensor 100 can be incorporated into the final joint system to provide data post-operatively to determine if the implanted joint is functioning correctly. Early determination of a problem using sensor 100 can reduce catastrophic failure of the joint that a patient is unaware of or cannot feel. The problem can often be fixed with a minimal invasive procedure at lower cost and stress to the patient. Similarly, longer term monitoring of the joint can determine wear or misalignment that if detected early can be adjusted for optimal life or replacement of a wear surface with minor surgery thereby extending the life of the implant. In general, sensor 100 can be shaped such that it can placed or engaged or affixed to or within load bearing surfaces used in any orthopedic applications related to the musculoskeletal system, joints, and tools associated therewith. Sensor 100 can provide information on a combination of one or more performance parameters of interest such as wear, stress, kinematics, kinetics, fixation strength, ligament balance, anatomic fit and longevity.

FIG. 2 is a simplified cross-sectional view of a sensing module 101 (or assemblage) in accordance with an exemplary embodiment. The sensing module (or assemblage) is an electro-mechanical assembly comprising electrical components and mechanical components that when configured and operated in accordance with a sensing mode performs as a positive feedback closed-loop measurement system. The measurement system can precisely measure applied forces, such as loading, on the electro-mechanical assembly.

In one embodiment, the electrical components can include ultrasound resonators or transducers, ultrasound waveguides, and signal processing electronics, but are not limited to these. The mechanical components can include biasing springs 32, spring retainers and posts, and load platforms 6, but are not limited to these. The electrical components and mechanical components can be inter-assembled (or integrated) onto a printed circuit board 36 to operate as a coherent ultrasonic measurement system within sensing module 101 and according to the sensing mode. As will be explained hereinbelow in more detail, the signal processing electronics 10 incorporate edge detect circuitry that detects an edge of a signal after it has propagated through waveguide 5. The detection initiates the generation of a new pulse by an ultrasound resonator or transducer that is coupled to waveguide 5 for propagation therethrough. Any change in transit time of a pulse through waveguide 5 is measured and correlates to a change in material property of waveguide 5. An external condition being applied to sensing module 101 such as pressure modifies waveguide 5 such that a corresponding change in material property is produced. An example is an applied pressure modifies the length of waveguide 5. Changes in length can be measured by sensor 101 and converted to pressure using known characteristics of the medium that waveguide 5 comprises.

Sensing module 101 comprises one or more assemblages 3 each comprised of one or more ultrasound resonators. As illustrated, waveguide 5 is coupled between a transducer 4 and a reflective surface 30. In general, reflective surface 30 has a significant acoustic impedance mismatch such that a pulsed energy wave is reflected from surface 30. Very little or none of the pulsed energy wave is transmitted through reflective surface 30 due to the acoustic impedance mismatch. In a non-limiting example, reflective surface 30 can comprise materials such as a polymer, plastic, metal such as steel, or polycarbonate. Transducer 4 and reflective surface 30 are affixed to load bearing or contacting surfaces 6 to which an external condition is applied. In one exemplary embodiment, an ultrasound signal is coupled for propagation through waveguide 5. The sensing module 101 is placed, attached to, or affixed to, or within a body, instrument, or other physical system 7 having a member or members 8 in contact with the load bearing or contacting surfaces 6 of the sensing module 101. This arrangement facilitates translating the parameters of interest into changes in the length or compression or extension of the waveguide or waveguides 5 within the sensing module or device 100 and converting these changes in length into electrical signals. This facilitates capturing data, measuring parameters of interest digitizing the data, and subsequently communicating that data through antenna 34 to external equipment with minimal disturbance to the operation of the body, instrument, appliance, vehicle, equipment, or physical system 7 for a wide range of applications.

The sensing module 101 supports three modes of operation: pulse propagation and measurement: reflectance, unidirectional, and bi-directional. These modes can be used as appropriate for each individual application. In unidirectional and bi-directional modes, a chosen ultrasound resonator or transducer is controlled to emit pulses of ultrasound waves into the ultrasound waveguide and one or more other ultrasound resonators or transducers are controlled to detect the propagation of the pulses of ultrasound waves at a specified location or locations within the ultrasound waveguide. In at least one exemplary embodiment, reflectance also described as pulse-echo mode is utilized. Pulse-echo mode uses a single transducer to emit pulsed energy waves into waveguide 5 and the single transducer subsequently detects pulses of echo waves after reflection from a selected feature or termination of the waveguide. In a non-limiting example, the pulsed energy wavers are ultrasound waves. In pulse-echo mode, echoes of the pulses can be detected by controlling the actions of an emitting ultrasound resonator or transducer to alternate between emitting and detecting modes of operation. Pulse and pulse-echo modes of operation may require operation with more than one emitted pulsed energy waves propagating within the waveguide at equilibrium.

Many parameters of interest within physical systems or bodies can be measured by evaluating changes in the transit time of energy pulses. The type and frequency of the energy pulse is determined by factors such as distance of measurement, medium in which the signal travels, accuracy required by the measurement, form factor of system, power constraints, and cost. In the non-limiting example, pulses of ultrasound energy provide accurate markers for measuring transit time of the pulses within waveguide 5. In general, an ultrasonic signal is an acoustic signal having a frequency above the human hearing range (e.g. >20 KHz). In one embodiment, a change in transit time of an ultrasonic energy pulse corresponds to a difference in the physical dimension of the waveguide from a previous state. For example, a force or pressure applied across the knee joint compresses wave guide 5 to a new length that is related to transit time of the energy pulse When integrated as a sensing module and inserted or coupled to a physical system or body, these changes are directly correlated to the physical changes on the system or body and can be readily converted to a pressure or a force.

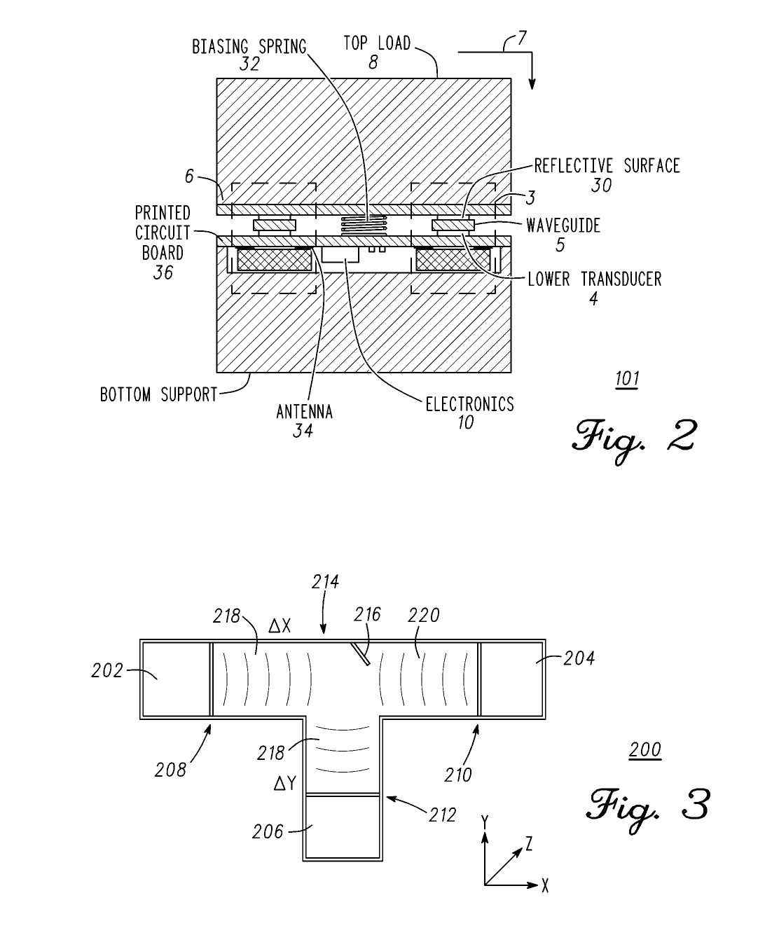

FIG. 3 is an exemplary assemblage 200 for illustrating reflectance and unidirectional modes of operation. It comprises one or more transducers 202, 204, and 206, one or more waveguides 214, and one or more optional reflecting surfaces 216. The assemblage 200 illustrates propagation of ultrasound waves 218 within the waveguide 214 in the reflectance and unidirectional modes of operation. Either ultrasound resonator or transducer 202 and 204 in combination with interfacing material or materials 208 and 210 can be selected to emit ultrasound waves 218 into the waveguide 214.

In unidirectional mode, either of the ultrasound resonators or transducers for example 202 is controlled to emit ultrasound waves 218 into the waveguide 214. The other ultrasound resonator or transducer 204 is controlled to detect the ultrasound waves 218 emitted by the emitting ultrasound resonator 202 or transducer.

In reflectance mode, the ultrasound waves 218 are detected by the emitting ultrasound resonator or transducer after reflection 220 from the opposite end of the waveguide 214 by a reflective surface, interface, or body at the opposite end of the waveguide. In this mode, either of the ultrasound resonators or transducers 202 or 204 can be selected to emit and detect ultrasound waves.

Additional reflection features 216 can be added within the waveguide structure to reflect ultrasound waves. This can support operation in a combination of unidirectional and reflectance modes. In this mode of operation, one of the ultrasound resonators, for example resonator 202 is controlled to emit ultrasound waves 218 into the waveguide 214. Another ultrasound resonator or transducer 206 is controlled to detect the ultrasound waves 218 emitted by the emitting ultrasound resonator 202 (or transducer) subsequent to their reflection by reflecting feature 216.

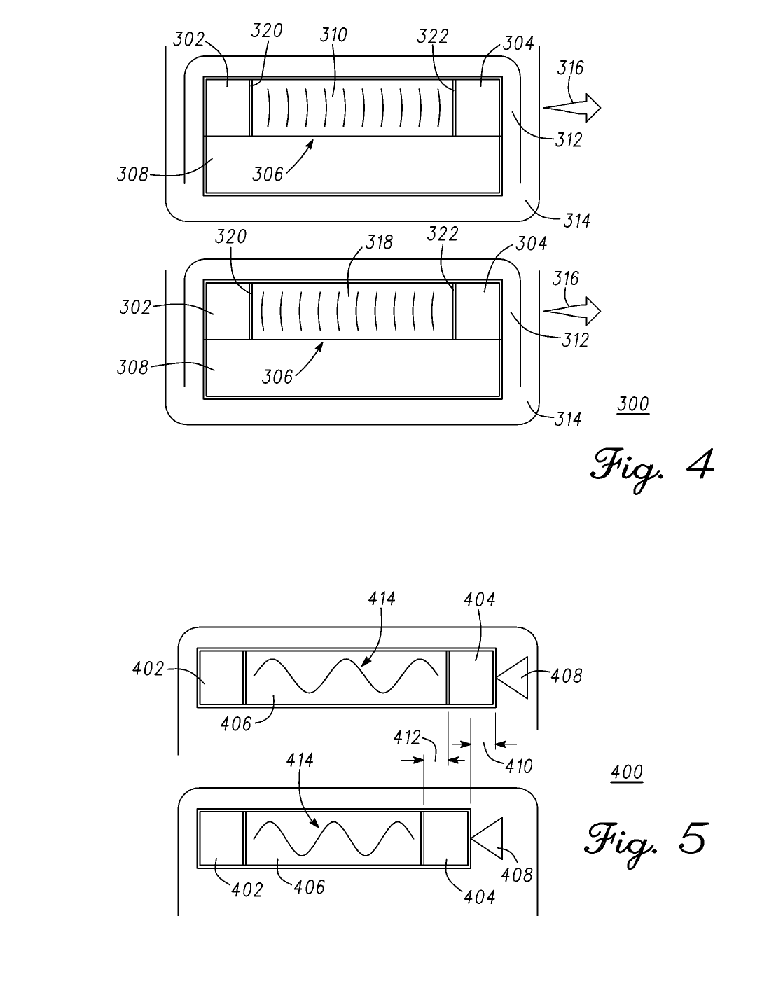

FIG. 4 is an exemplary assemblage 300 that illustrates propagation of ultrasound waves 310 within the waveguide 306 in the bi-directional mode of operation of this assemblage. In this mode, the selection of the roles of the two individual ultrasound resonators (302, 304) or transducers affixed to interfacing material 320 and 322 are periodically reversed. In this mode the transit time of ultrasound waves propagating in either direction within the waveguide 306 can be measured. This can enable adjustment for Doppler effects in applications where the sensing module 308 is operating while in motion 316. Furthermore, this mode of operation helps assure accurate measurement of the applied load, force, pressure, or displacement by capturing data for computing adjustments to offset this external motion 316. An advantage is provided in situations wherein the body, instrument, appliance, vehicle, equipment, or other physical system 314, is itself operating or moving during sensing of load, pressure, or displacement. Similarly, the capability can also correct in situation where the body, instrument, appliance, vehicle, equipment, or other physical system, is causing the portion 312 of the body, instrument, appliance, vehicle, equipment, or other physical system being measured to be in motion 316 during sensing of load, force, pressure, or displacement. Other adjustments to the measurement for physical changes to system 314 are contemplated and can be compensated for in a similar fashion. For example, temperature of system 314 can be measured and a lookup table or equation having a relationship of temperature versus transit time can be used to normalize measurements. Differential measurement techniques can also be used to cancel a common factor as is known in the art.

The use of waveguide 306 enables the construction of low cost sensing modules and devices over a wide range of sizes, including highly compact sensing modules, disposable modules for bio-medical applications, and devices, using standard components and manufacturing processes. The flexibility to construct sensing modules and devices with very high levels of measurement accuracy, repeatability, and resolution that can scale over a wide range of sizes enables sensing modules and devices to the tailored to fit and collect data on the physical parameter or parameters of interest a wide range of medical and non-medical applications.

For example, sensing modules or devices may be placed on or within, or attached or affixed to or within, a wide range of physical systems including, but not limited to instruments, appliances, vehicles, equipments, or other physical systems as well as animal and human bodies, for sensing the parameter or parameters of interest in real time without disturbing the operation of the body, instrument, appliance, vehicle, equipment, or physical system.

In addition to non-medical applications, examples of a wide range of potential medical applications may include, but are not limited to, implantable devices, modules within implantable devices, modules or devices within intra-operative implants or trial inserts, modules within inserted or ingested devices, modules within wearable devices, modules within handheld devices, modules within instruments, appliances, equipment, or accessories of all of these, or disposables within implants, trial inserts, inserted or ingested devices, wearable devices, handheld devices, instruments, appliances, equipment, or accessories to these devices, instruments, appliances, or equipment. Many physiological parameters within animal or human bodies may be measured including, but not limited to, loading within individual joints, bone density, movement, various parameters of interstitial fluids including, but not limited to, viscosity, pressure, and localized temperature with applications throughout the vascular, lymph, respiratory, and digestive systems, as well as within or affecting muscles, bones, joints, and soft tissue areas. For example, in orthopedic applications this may include, but is not limited to, load bearing prosthetic components, or provisional or trial prosthetic components for, but not limited to, surgical procedures for knees, hips, shoulders, elbows, wrists, ankles, and spines; any other orthopedic or musculoskeletal implant, or any combination of these.

FIG. 5 is an exemplary cross-sectional view of a sensor element 400 to illustrate changes in the propagation of ultrasound waves 414 with changes in the length of a waveguide 406. An external force 408 compresses waveguide 406 thereby changing the length of waveguide 406. Sensing circuitry (not shown) measures propagation characteristics of ultrasonic signals in the waveguide 406 to determine the change in the length of the waveguide 406. These changes in length change in direct proportion to the parameters of interest thus enabling the conversion of changes in the parameter or parameters of interest into electrical signals.

As illustrated, external force 408 compresses waveguide 406 and pushes the transducers 402 and 404 closer to one another by a distance 410. This changes the length 412 of the waveguide propagation path between transducers 402 and 404. Depending on the operating mode, the sensing circuitry measures the change in length of the waveguide 406 by analyzing characteristics of the propagation of ultrasound waves within the waveguide.

One interpretation of FIG. 5 illustrates waves emitting from transducer 402 at one end of waveguide 406 and propagating to transducer 404 at the other end of the waveguide 406. The interpretation includes the effect of movement of waveguide 406 and thus the velocity of waves propagating within waveguide 406 (without changing shape or width of individual waves) and therefore the transit time between transducers 402 and 404 at each end of the waveguide. The interpretation further includes the opposite effect on waves propagating in the opposite direction and is evaluated to estimate the velocity of the waveguide and remove it by averaging the transit time of waves propagating in both directions in turns, not simultaneously.

Changes in the parameter or parameters of interest are measured by measuring changes in the transit time of energy pulses or waves within the propagating medium. Closed loop measurement of changes in the parameter or parameters of interest is achieved by modulating the repetition rate of energy pulses or the frequency of energy waves as a function of the propagation characteristics of the elastic energy propagating structure.

These measurements may be implemented with an integrated wireless sensing module or device having an encapsulating structure that supports sensors and load bearing or contacting surfaces and an electronic assemblage that integrates a power supply, sensing elements, energy transducer or transducers and elastic energy propagating structure or structures, biasing spring or springs or other form of elastic members, an accelerometer, antennas and electronic circuitry that processes measurement data as well as controls all operations of ultrasound generation, propagation, and detection and wireless communications. The electronics assemblage also supports testability and calibration features that assure the quality, accuracy, and reliability of the completed wireless sensing module or device.

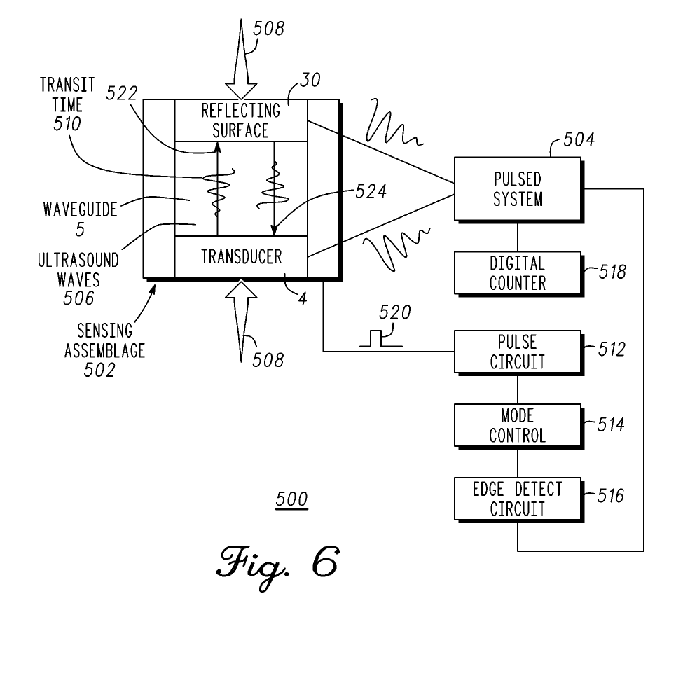

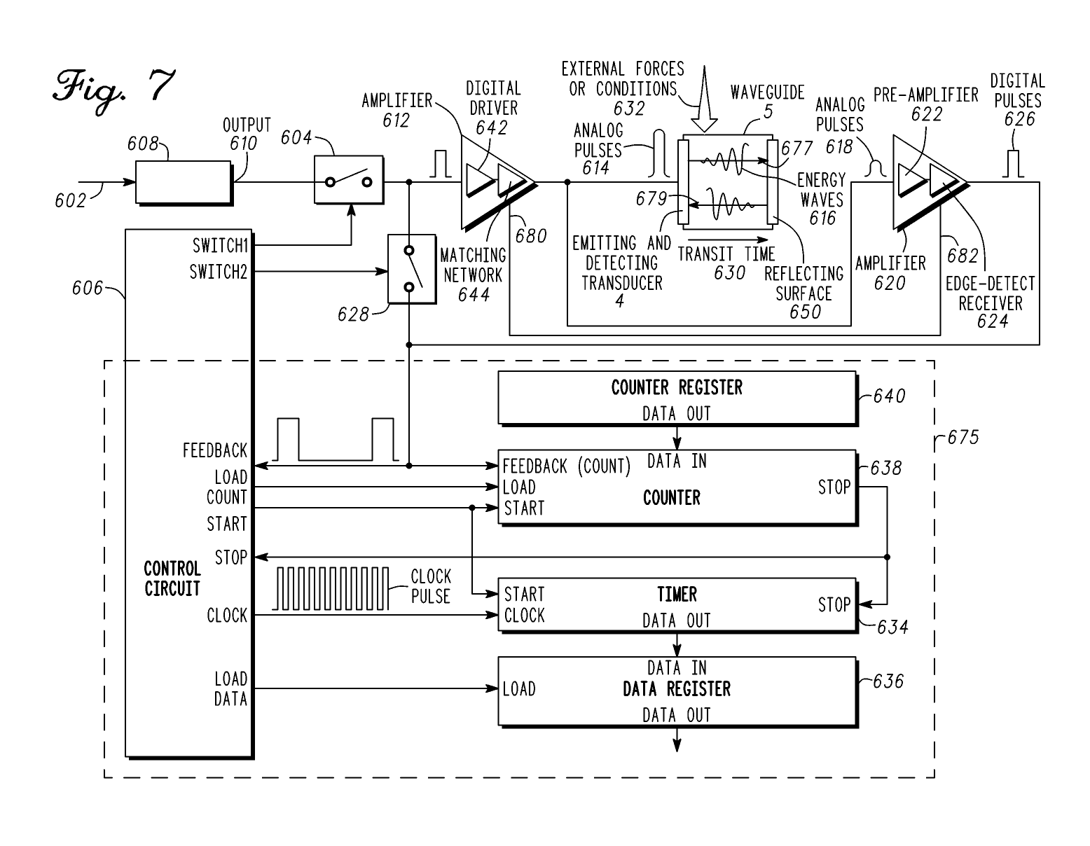

FIG. 6 is an exemplary block diagram 500 of a measurement system in accordance with one embodiment. The measurement system comprises components of the sensing module 101 shown in FIG. 2. The measurement system includes a sensing assemblage 502 and a pulsed system 504 that detects energy waves 506 in one or more waveguides 5 of the sensing assembly 502. A pulse 520 is generated in response to the detection of energy waves 506 to initiate a propagation of a new pulse in waveguide 5.

The sensing assembly 502 comprises transducer 4, reflective surface 30, and a waveguide 5 (or energy propagating structure). In a non-limiting example, sensing assemblage 502 is affixed to load bearing or contacting surfaces 508. External forces or conditions for measurement are applied to the contacting surfaces 508. In at least one exemplary embodiment, the external forces 508 compress the waveguide 5 thereby changing the length of the waveguide 5 depending on the force applied thereon. Similarly, transducer 4 and reflective surface 30 move closer together under compression. In the reflected or pulsed echo mode, a transit time 510 of a pulsed energy wave comprises a time period indicated by arrow 522 of the pulsed energy wave moving from transducer 4 through waveguide 5 to reflective surface 30 plus the echo time period indicated by arrow 524 comprising a reflected pulse energy wave moving from reflective surface 30 through waveguide 5 back to transducer 4. Thus, a change in length of waveguide 5 affects the transit time 510 of energy waves 506 comprising the transmitted and reflected path. The pulsed system 504 in response to these physical changes will detect each energy wave sooner (e.g. shorter transit time) and initiate the propagation of new pulses associated with the shorter transit time. As will be explained below, this is accomplished by way of pulse system 504 in conjunction with the pulse circuit 512, the mode control 514, and the edge detect circuit 516.

Notably, changes in the waveguide 5 (energy propagating structure or structures) alter the propagation properties of the medium of propagation (e.g. transmit time 510). A pulsed approach reduces power dissipation allowing for a temporary power source such as a battery or capacitor to power the system during the course of operation. In at least one exemplary embodiment, a pulse is provided to transducer 4 coupled to a first surface of waveguide 5. Transducer 4 generates a pulsed energy wave 506 coupled into waveguide 5. In a non-limiting example, transducer 4 is a piezo-electric device capable of transmitting and receiving acoustic signals in the ultrasonic frequency range. Transducer 4 is toggled between an emitting mode to emit a pulsed energy wave into waveguide 5 and a receiving mode to generate an electrical signal corresponding to a reflected pulsed energy wave.

In a start up mode, transducer 4 is enabled for receiving the reflected pulsed energy wave after generating one or more pulsed energy waves and delivering them into waveguide 5. Upon receiving the reflected pulsed energy wave, transducer 4 generates an electrical signal corresponding to the reflected pulsed energy wave. The electrical signal output by transducer 4 is coupled to edge detect circuit 516. In at least one exemplary embodiment, edge detect circuit 516 detects a leading edge of the electrical signal output by transducer 4 (e.g. the propagated reflected energy wave 506). The detection of the reflected propagated pulsed signal occurs earlier (due to the length/distance reduction of waveguide 5) than a prior signal due to external forces 508 being applied to compress sensing assemblage 502. Pulse circuit 512 generates a new pulse in response to detection of the propagated and reflected pulsed signal by edge detect circuit 516. Transducer 4 is then enabled to generate a new pulsed energy wave. A pulse from pulse circuit 512 is provided to transducer 4 to initiate a new pulsed sequence. Thus, each pulsed sequence is an event of pulse propagation, pulse detection and subsequent pulse generation that initiates the next pulse sequence.

The transit time 510 of the propagated pulse is the total time it takes for a pulsed energy wave to travel from transducer 4 to reflecting surface 30 and from reflecting surface 30 back to transducer 4. There is delay associated with each circuit described above. Typically, the total delay of the circuitry is less than the propagation time of a pulsed signal through waveguide 5. Multiple pulse to pulse timings can be used to generate an average time period when change in external forces 508 occur relatively slowly in relation to the pulsed signal propagation time. The digital counter 518 in conjunction with electronic components counts the number of propagated pulses to determine a corresponding change in the length of the waveguide 5. These changes in length change in direct proportion to the external force thus enabling the conversion of changes in parameter or parameters of interest into electrical signals.

In at least one exemplary embodiment, pulsed system 504 in conjunction with one or more sensing assemblages 502 are used to take measurements on a muscular-skeletal system. In a non-limiting example, sensing assemblage 502 is placed between a femoral prosthetic component and tibial prosthetic component to provide measured load information that aids in the installation of an artificial knee joint. The measurements can be made in extension and in flexion. Assemblage 502 is used to measure the condyle loading to determine if it falls within a predetermined range. Based on the measurement, the surgeon can select the thickness of the insert such that the measured loading with the final insert in place will fall within the predetermined range. Soft tissue tensioning can be used by a surgeon to further optimize the force or pressure. Similarly, two assemblages 502 can be used to measure both condyles simultaneously or multiplexed. The difference in loading (e.g. balance) between condyles can be measured. Soft tissue tensioning can be used to reduce the force on the condyle having the higher measured loading to reduce the measured pressure difference between condyles.

One method of operation holds the number of pulsed energy waves propagating through waveguide 5 as a constant integer number. A time period of a pulsed energy wave corresponds to the time between the leading pulse edges of adjacent pulsed energy waves. A stable time period or a period of equilibrium is one in which the time period changes very little over a number of pulsed energy waves. This occurs when conditions that affect sensing assemblage 502 stay consistent or constant. Holding the number of pulsed energy waves propagating through waveguide 5 to an integer number is a constraint that forces a change in the time between pulses when the length of waveguide 5 changes. The resulting change in time period of each pulsed energy wave corresponds to a change in aggregate pulse periods that can be captured using digital counter 518 as a measurement of changes in external forces or conditions 508.

In an alternate embodiment, the repetition rate of pulsed energy waves 506 emitted by transducer 4 can be controlled by pulse circuit 512. The operation remains similar where the parameter to be measured corresponds to the measurement of the transit time 510 of pulsed energy waves 506 within waveguide 5 as described above. It should be noted that an individual ultrasonic pulse can comprise one or more energy waves with a damping wave shape as shown. The pulsed energy wave shape is determined by the electrical and mechanical parameters of pulse circuit 512, interface material or materials, where required, and ultrasound resonator or transducer 4. The frequency of the pulsed energy waves is determined by the response of the emitting ultrasound resonator 4 to excitation by an electrical pulse 520. The mode of the propagation of the pulsed energy waves 506 through waveguide 5 is controlled by mode control circuitry 514 (e.g., reflectance or uni-directional). The detecting ultrasound resonator or transducer may either be a separate ultrasound resonator or the emitting resonator or transducer 4 depending on the selected mode of propagation (reflectance or unidirectional).

In general, accurate measurement of physical parameters is achieved at an equilibrium point having the property that an integer number of pulses are propagating through the energy propagating structure at any point in time. Measurement of changes in the “time-of-flight” or transit time of ultrasound pulses within a waveguide of known length can be achieved by modulating the repetition rate of the ultrasound pulses as a function of changes in distance or velocity through the medium of propagation, or a combination of changes in distance and velocity, caused by changes in the parameter or parameters of interest

It should be noted that ultrasound energy pulses or waves, the emission of ultrasound pulses or waves by ultrasound resonators or transducers, transmitted through ultrasound waveguides, and detected by ultrasound resonators or transducers are used merely as examples of energy pulses, waves, and propagation structures and media. Other embodiments herein contemplated can utilize other wave forms, such as, light.

Measurement by pulsed system 504 and sensing assemblage 502 enables high sensitivity and signal-to-noise ratio as the time-based measurements are largely insensitive to most sources of error that may influence voltage or current driven sensing methods and devices. The resulting changes in the transit time of operation corresponds to frequency which can be measured rapidly and with high resolution. This achieves the required measurement accuracy and precision thus capturing changes in the physical parameters of interest and enabling analysis of their dynamic and static behavior.

FIG. 7 is a measurement system operating in pulsed echo mode with digital output according to one embodiment. In particular, with respect to FIG. 6, it illustrates positive feedback closed-loop measurement of the transit time 510 of pulsed energy waves 506 within the waveguide 5 by the operation of pulsed system 504. A pulsed echo mode is one of the modes of operation of the system. In pulsed echo mode, a pulsed energy wave is provided by emitting transducer 4, propagated through waveguide 5 (e.g. propagating structure), reflected by reflecting surface 650, and the reflected pulse energy wave is received by transducer 4. Briefly, the digital logic circuit 675 digitizes the frequency of operation of the pulsed system 504.

Referring to FIG. 2, in pulse echo mode of operation, the sensing module 101 measures a time of flight (TOF) of a pulsed energy wave transmitted by transducer 4 into waveguide 5, reflected, and received by transducer 4. The time of flight determines the length of the waveguide propagating path, and accordingly reveals the change in length of the waveguide 5 due to a parameter applied thereto. In another arrangement, differential time of flight measurements can be used to determine the change in length of the waveguide 5. A pulse can comprise a pulse of one or more waves. The waves may have equal amplitude and frequency (square wave pulse) or they may have different amplitudes, for example, decaying amplitude (trapezoidal pulse) or some other complex waveform. The pulsed system detects an edge of each pulse propagating through the waveguide and holds the delay between each edge constant under stable operating conditions.

A pulse method facilitates separation of ultrasound frequency, damping waveform shape, and repetition rate of pulses of ultrasound waves. Separating ultrasound frequency, damping waveform shape, and repetition rate enables operation of ultrasound transducers at or near resonance to achieve higher levels of conversion efficiency and power output thus achieving efficient conversion of ultrasound energy. This may enable, but is not limited to, lower power operation for ultra-low power devices.

In a non-limiting example, pulse echo mode operation is initiated with control circuitry 606 closing switch 604, which couples an output 610 of pulse circuit 608 to an input of amplifier 612. Pulse circuit 608 initializes the circuit by sending one or more pulses to amplifier 612. Pulse circuit 608 can be enabled for providing pulses by control circuit 606. Amplifier 612 provides analog pulses 614 to an input of transducer 4. Amplifier 612 having digital driver 642 and matching network 644 transforms the digital output 610 (e.g. square wave) of pulse circuit 608 into analog pulses 614 that are modified for emitting transducer 4. The repetition rate of analog pulses 614 is substantially equal to the pulses at output 610 of pulse circuit 612. Amplifier 612 drives transducer 4 with sufficient power to generate energy waves 616. In at least one exemplary embodiment, transducer 4 converts the pulsed electrical waves into pulsed energy waves 616 having the same repetition rate and emits them into energy propagating structure or waveguide 5. In a non-limiting example, energy waves 616 are ultrasound waves.

In general, ultrasound transducers naturally resonate at a predetermined frequency. Providing a square wave to the input of emitting transducer 4 could yield undesirable results. Digital driver 642 of amplifier 612 drives matching network 644. Matching network 644 is optimized to match an input impedance of emitting transducer 4 for efficient power transfer. In at least one exemplary embodiment, digital driver 642, matching network 644, solely, or in combination shapes or filters pulses provided to the input of amplifier 612. The waveform is modified from a square wave to analog pulse 614 to minimize ringing and to aid in the generation of a damped waveform by emitting transducer 4. In one embodiment, the pulsed energy wave emitted into waveguide 5 can ring and has a damped envelope that affects signal detection which will be disclosed in more detail below.

The one or more pulsed energy waves 616 propagate through energy propagating structure or medium 5. The one or more pulsed energy waves 616 propagate in a direction indicated by arrow 677. Pulsed energy waves 616 are reflected by reflecting surface 650. The reflected pulse energy waves propagate in a direction indicated by arrow 679. As shown, reflected pulsed energy waves propagate towards transducer 4. In general, pulsed energy waves in medium 5 traverse the length of the propagating structure twice. Thus, any measured change and subsequent conversion to a measured parameter takes the fact that the propagation distance is twice the length of waveguide 5 into account.

Transducer 4 has two modes of operation comprising emitting and receiving a pulsed energy wave. In one embodiment, amplifier 620 is decoupled or blanked when transducer 4 is in an emitting mode. A terminal 680 of amplifier 612 is coupled to a terminal 682 of amplifier 620. A signal is provided by amplifier 612 in response to a received pulse, the signal is output at terminal 682 to decouple or blank amplifier 620. Blanking or decoupling amplifier 620 prevents amplifier 620 from generating a pulse in response to a signal output by amplifier 612. The signal from amplifier 612 enables amplifier 620 to receive or detect a reflected pulsed energy wave propagating through waveguide 5 from reflecting surface 650 after amplifier 612 has provided analog pulse 614 to emit a pulsed energy wave into the propagating structure.

Amplifier 620 comprises pre-amplifier 622 and edge-detect receiver 624. Pre-amplifier 622 is coupled to transducer 4. Preamplifier 622 receives and amplifies analog pulses 618 from transducer 4 and provides the amplified signal to edge-detect receiver 624. Edge-detect receiver 624 detects an edge of each analog pulse corresponding to each propagated pulsed energy wave through waveguide 5. As mentioned previously, each pulsed energy wave can be a ringing damped waveform. In at least one exemplary embodiment, edge-detect receiver 626 detects a leading edge of each analog pulse 618. Edge-detect receiver 626 can have a threshold such that signals below the threshold cannot be detected. Edge-detect receiver 626 can include a sample and hold that prevents triggering on subsequent edges of a ringing damped signal. The sample and hold can be designed to “hold” for a period of time where the damped signal will fall below the threshold but less than the shortest edge to edge time period between adjacent pulsed energy waves under all operating conditions. Amplifier 620 generates a digital pulse 624 triggered off each leading edge of each propagated pulsed energy wave detected by transducer 4. Each digital pulse 624 is of sufficient length to sustain the pulse behavior of the closed loop circuit as it is coupled back to amplifier 612.

Control circuitry 606 responds to receiving a first digital output pulses 626 from amplifier 620 by closing switch 628 and opening switch 604. Closing switch 628 creates a positive feedback closed loop circuit coupling a pulse generated by amplifier 620 to the input of amplifier 612 and sustaining a sequence of generated pulsed energy wave emission into wave guide 5, propagation of the pulsed energy wave through waveguide 5, reflecting the pulsed energy wave off reflecting surface 650, and detection of the reflected pulsed energy wave after traveling back through waveguide 5, and generation of a new digital pulses 626.

In a pulsed echo mode of operation, transducer 4 toggles back and forth from emitting a pulsed energy wave into waveguide 5 and receiving a reflected pulsed energy wave. Upon receiving the reflected pulse energy wave, transducer 4 converts the reflected pulsed energy wave into an electrical signal that is output as analog pulses 618 having the same repetition rate. Transducer 4 subsequently emits a new pulsed energy wave into waveguide 5 in response to analog pulses 614 provided by amplifier 612. Thus, transducer 4 is used in a repeating sequence of emitting and detecting. The analog pulses 618 output by transducer 4 (in the reflected pulse receiving mode) may need amplification.

In one embodiment, the delay of amplifier 620 and 612 is small in comparison to the propagation time of a pulsed energy wave through waveguide 5. In an equilibrium state, an integer number of pulses of energy waves 616 propagate through energy propagating structure or waveguide 5. For example, a single pulsed energy wave propagates through waveguide 5. As one energy pulse wave exits waveguide 5, a new energy pulse wave is emitted into waveguide 5 that is delayed by the combined signal generation time of amplifier 620 and amplifier 612. Movement or changes in the physical properties of the energy propagating structure or waveguide 5 change the transit time 630 of energy waves 616. This disrupts the equilibrium thereby changing when a pulsed energy wave is detected by edge-detect receiver 626. For example, the transit time 630 is reduced should external forces 632 compress waveguide 5. Conversely, the transit time 630 is increased should external forces 632 result in waveguide 5 expanding. The change in transit time 630 delivers digital pulses 624 earlier or later than previous pulses thereby producing an adjustment to the delivery of analog pulses 618 and 614 to a new equilibrium point. The new equilibrium point will correspond to a different transit time (e.g. different instantaneous frequency) but the same integer number of pulses. As disclosed above in pulsed echo mode, transit time 630 comprises the time for a pulsed energy wave to propagate from transducer 4 to reflecting surface 650 plus the time for the reflected pulsed energy wave to propagate from reflecting surface 650 to transducer 4.

As previously disclosed, the repetition rate of energy waves 616 during operation of the closed loop circuit, and changes in this repetition rate, can be used to measure changes in the movement or changes in the physical attributes of energy propagating structure or medium 5. The changes can be imposed on the energy propagating structure or medium 5 by external forces or conditions 632 thus translating the levels and changes of the parameter or parameters of interest into signals that may be digitized for subsequent processing, storage, and display. In at least one exemplary embodiment, the external forces 632 compress the waveguide 5 in a direction of the travel of the pulsed energy waves thereby changing the distance traversed. The length of waveguide 5 corresponds to the pressure applied. Thus, the frequency of energy waves 616 can be related to a pulsed energy wave time period of single pulsed energy wave or over multiple pulsed energy wave time periods during the operation of the closed loop circuit, and changes in this frequency, can be used to measure movement or changes in physical attributes of energy propagating structure or medium 5.

The changes in physical attributes of energy propagating structure or medium 5 by external forces or conditions 632 translates the levels and modifies the parameter or parameters of interest into a time period difference of adjacent pulses, a time period difference of transit time 630, or a difference averaged over multiple time periods for the pulsed energy wave time period or transit time 630. The time period or transit time 630 corresponds to a frequency for the time period measured. The new frequency can be digitized for subsequent transmission, processing, storage, and display. Translation of the measured frequency into digital binary numbers facilitates communication, additional processing, storage, and display of information about the level and changes in physical parameters of interest. Prior to measurement of the frequency, control logic 606 loads the loop count into digital counter 634 that is stored in digital register 636.

Digital logic circuit 675 is described in more detail hereinbelow. As previously mentioned, a first pulse from digital pulses 624 initiates a parameter measurement or sensing of waveguide 5. In at least one exemplary embodiment, sensing does not occur until initial equilibrium has been established. Alternatively, each time period of a pulsed energy wave or transit time period 630 of the pulsed energy wave can be measured and reviewed. Control circuit 606 detects digital pulses 626 from amplifier 620 (closing switch 628 and opening switch 604) to establish equilibrium and start measurement operations. In an extended configuration of pulse echo mode, a digital block is coupled to the pulsed echo mode measurement system for digitizing the frequency of operation. Translation of the time period of pulsed energy waves into frequency (digital binary numbers) facilitates communication, additional processing, storage, and display of information about the level and changes in physical parameters of interest. During this process, control circuit 606 enables digital counter 638 and digital timer 634. Digital counter 638 decrements its value on the rising edge of each digital pulse output by amplifier 620. Digital timer 634 increments its value on each rising edge of pulses from oscillator output 610. A clock such as a crystal oscillator is used to clock digital logic circuit 675 and as a reference in which to gauge time periods of pulsed energy waves. Alternatively, pulse circuit 608 can be a reference clock. When the number of digital pulses 626 has decremented the value within digital counter 638 to zero a stop signal is output from digital counter 638. The stop signal disables digital timer 634 and triggers control logic 606 to output a load command to data register 636. Data register 636 loads a binary number from digital timer 634 that is equal to the period of the energy waves or pulses times the value in counter 638 divided by a clock period corresponding to oscillator output 610. With a constant clock period, the value in data register 636 is directly proportional to the aggregate period of the pulsed energy waves or pulses accumulated during the measurement operation. Duration of the measurement operation and the resolution of measurements may be adjusted by increasing or decreasing the value preset in the count register 640.

This method of operation further enables setting the level of precision or resolution of the captured data by using long cycle counts to optimize trade-offs between measurement resolution versus pulse repetition rate, ultrasound frequency, and damping waveform shape, as well as the bandwidth of the sensing and the speed of the data processing operations to achieve an optimal operating point for a sensing module or device.

In at least one exemplary embodiment, the sensor system includes the system as a wireless module that operates according to one or more criteria such as, but not limited to, power level, applied force level, standby mode, application context, temperature, or other parameter level. Pulse shaping can also be applied to increase reception quality depending on the operational criteria. The wireless sensing module comprises the pulsed measurement system, one or more sensing assemblies, one or more load surfaces, an accelerometer, electronic circuitry, a transceiver, and an energy supply. The wireless sensing module measures a parameter such as force/pressure and transmits the measurement data to a secondary system for further processing and display. The electronic circuitry in conjunction with the sensing assemblies accurately measures physical displacements of the load surfaces on the order of a few microns along various physical dimensions. The sensing assembly physically changes in response to an applied force, such as an applied load. Electronic circuitry operating in a positive feedback closed-loop circuit configuration precisely measures changes in propagation time due to changes in the length of the waveguides; physical length changes which occur in direct proportion to the applied force.

Upon reviewing the aforementioned embodiments, it would be evident to an artisan with ordinary skill in the art that said embodiments can be modified, reduced, or enhanced without departing from the scope and spirit of the claims described below. As an example:

Changing repetition rate of complex waveforms to measure time delays.

Changing repetition rate of acoustical, sonic, or light, ultraviolet, infrared, RF or other electromagnetic waves, pulses, or echoes of pulses to measure changes in the parameter or parameters of interest.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. A pulsed echo mode measurement system comprising:
one or more sensing assemblies;
a pulsed system;
one or more load surfaces; and electronic circuitry, where the pulsed system maintains positive closed-loop feedback of pulsed energy waves in one or more energy propagating structures of the sensing assembly, where the system measures parameters of the muscular-skeletal system and where the pulsed energy waves are reflected at least once in the one or more energy propagating structures.

2. The system of claim 1, where the pulsed system modulates a time period of pulsed energy waves as a function of changes in distance or velocity through a medium of the one or more energy propagating structures, or a combination of changes in distance and velocity, caused by changes in the one or more energy propagating structures.

3. The system of claim 1, further comprising a pulse shaper to dampen a wave shape for optimal transmission and reception in accordance with a matched network.

4. The system of claim 1, further comprising a digital block for digitizing the frequency of operation of the pulsed system.

5. The system of claim 1 where the pulsed system is configured to operate wireless in pulsed echo mode according to one or more operational criteria, such as, but not limited to, power level, applied force level, standby mode, application context, temperature, or other parameter level.

6. The system of claim 5, where the system operates to measure changes in transit time due to changes in the length of one or more waveguides coupled to the one or more load surfaces such that the physical length changes under load are in proportion to the applied force.

7. A sensor module comprising one or more sensors for sensing a muscular-skeletal system each sensor comprising:
a transducer;
a waveguide having a first surface and a second surface where the transducer couples to the first surface of the waveguide; and
a reflective surface coupled to the second surface of the waveguide where pulsed energy waves propagate through the waveguide and where a transit time of a pulsed energy wave through the wave guide corresponds to one or more measured parameters of the muscular-skeletal system.

8. The sensor module of claim 7 where a change in length of the waveguide results in a corresponding change in the transit time of the pulsed energy wave and where the transit time or a change in transit time in conjunction with material properties of the waveguide corresponds to the one or more measured parameters.

9. The sensor module of claim 7 where each pulsed energy wave is detected after propagating through the waveguide, where a pulse is generated when each pulsed energy wave is detected, and where the pulse is coupled to the first transducer to emit a pulsed energy wave into the waveguide.

10. The sensor module of claim 9 further including:
a first amplifier having an input and an output coupled to the transducer; and
a second amplifier having an input coupled to the transducer and an output coupled to the input of the first amplifier.

11. The sensor module of claim 7 where the waveguide comprises a polymer material.

12. The sensor module of claim 7 where the sensor module further includes:
a first load bearing surface having an external surface and an internal surface;
a second load bearing surface having an external surface and an internal surface where a stack is formed comprising:
the transducer coupled to the internal surface of the first load bearing surface;
the waveguide; and
the reflective surface where the second transducer is coupled to the internal surface of the second load bearing surface.

13. The sensor of module of claim 12 where the sensor module is coupled between an orthopedic joint to measure at least one of pressure, weight, strain, wear, vibration, density, temperature, or distance.

14. The sensor module of claim 12 further including at least one biasing spring coupled between the internal surface of the first and second load bearing surfaces.

15. A sensor comprising:
a first amplifier having an input and an output;
a transducer having a terminal coupled to the output of the first amplifier;
an energy wave propagation medium having a first surface coupled to the transducer and a second surface where the second surface is reflective; and
a second amplifier having an input coupled to the transducer and an output coupled to the input of the first amplifier where the sensor measures parameters of the muscular-skeletal system.

16. The sensor of claim 15 where one or more pulsed energy waves are provided to the input of the first amplifier to initiate sensing and where the second amplifier is decoupled from the input of the first amplifier during initialization.

17. The sensor of claim 16 where the second amplifier is coupled to the input of the first amplifier when the transducer receives a first reflected energy wave and where a time period of energy waves are substantially equal when conditions on the sensor remain constant.

18. The sensor of claim 17 where a transit time of an energy wave propagating through the medium corresponds to a parameter being measured and where a change in the medium due to the parameter being measured produces a corresponding change in the transit time.

19. The sensor of claim 18 where the transit time of energy waves propagating through the medium corresponds to one of pressure, weight, strain, wear, vibration, density, temperature, or distance.

20. The sensor of claim 15 where an integer number of energy waves couple through the medium under an equilibrium condition.

21. The sensor of claim 15 where the first amplifier comprises:
a digital driver having an input corresponding to the input of the first amplifier and an output; and
a matching network having an input coupled to the output of the digital driver and an output corresponding to the output of the first amplifier.

22. The sensor of claim 15 where the second amplifier comprises:
a preamplifier having an input corresponding to the input of the second amplifier and an output; and
an edge-detect receiver having an input coupled to the output of the preamplifier and an output corresponding to the output of the second amplifier.

23. The sensor of claim 15 further including:
a pulse circuit having an output for providing pulses of energy waves;
a first switch having a first terminal coupled to the output of the pulse circuit and a second terminal coupled to the input of the first amplifier where the first switch is closed to initiate the sensor and where the first switch is opened when an energy wave is detected by the second amplifier; and
a second switch having a first terminal coupled to the output of the second amplifier and a second terminal coupled to the input of the first amplifier where the first switch is open when the first switch is closed and where the second switch is closed when the first switch is open.
